(19) United States
(12) Patent Application Publication
(10) Pub. No.: US 2007/0184063 A1
(43) Pub. Date: Aug. 9, 2007

(54) Production of Proteins Carrying Oligomannose or Human-Like Glycans in Yeast and Methods of Use Thereof

(76) Inventors: Jan Holgersson, Huddinge (SE); Anki Gustafsson, Johanneshov (SE)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/626,156

(22) Filed: Jan. 23, 2007

Related U.S. Application Data
(60) Provisional application No. 60/761,632, filed on Jan. 23, 2006.

(51) Int. Cl. A61K 39/00; C07H 21/04; C12P 21/06; C07K 14/47; C12N 1/18

(52) U.S. Cl. 424/185.1; 435/69.1; 435/483; 435/254.23; 530/395; 536/23.5

(57) ABSTRACT

Cell lines having genetically modified glycosylation pathways that allow them to carry out a sequence of enzymatic reactions, which mimic the processing of glycoproteins in humans, have been developed. Recombinant proteins expressed in these engineered hosts yield glycoproteins more similar, if not substantially identical, to their human counterparts. The lower eukaryotes, which ordinarily produce high-mannose containing N-glycans, including unicellular and multicellular fungi are modified to produce O-glycans or other structures along human glycosylation pathways. This is achieved using a combination of engineering and/or selection of strains which: do not express certain enzymes which create the undesirable complex structures characteristic of the fungal glycoproteins, which express exogenous enzymes selected either to have optimal activity under the conditions present in the fungi where activity is desired, or which are targeted to an organelle where optimal activity is achieved, and combinations thereof wherein the genetically engineered eukaryote expresses multiple exogenous enzymes required to produce “human-like” glycoproteins.

### RELATED APPLICATIONS

This application claims the benefit of U.S. Ser. No. 60/761,632 filed Jan. 23, 2006, the contents of which is incorporated herein by reference in its entirety.

### FIELD OF THE INVENTION

The present invention relates to the field of glycoprotein production and protein glycosylation engineering in lower eukaryotes, specifically the production of glycoproteins in yeast having oligomannose or humanized O-glycans expressed. The present invention further relates to novel host cells comprising genes encoding enzymes involved in N-acetylgalactosamine transfer to serine or threonine in the peptide chain and production of glycoproteins that are particularly useful as therapeutic agents.

### BACKGROUND OF THE INVENTION

The possiblity of producing human recombinant proteins for therapy has revolutionized the treatment of patients with a variety of different diseases. Some proteins, for example insulin that is not glycosylated, can be produced in prokaryotic hosts such as E. coli. Most therapeutic proteins need to be modified by the addition of sugar residues to specific amino acids in the peptide sequence. This glycosylation may be necessary for correct folding of the protein, for long circulation half-times and, in many cases, for optimal activity of the protein. At present, glycosylated proteins are responsible for more than 60% of the annual turnover worldwide for therapeutic proteins. Mammalian cells can produce proteins with a human-like glycosylation, but have other disadvantages like low productivity, with regard to glycosylation heterogenous product formation, and the risk of virus contamination. Yeast cells are robust organisms for industrial fermentation and can be cultivated to high densities in well-defined media.

The glycosylation phenotype of glycoproteins produced in yeast is characterized by oligosaccharides with a high number of mannose residues. N-linked glycans of Pichia are mostly (˜85%) of the high mannose type containing between 8 and 14 mannose residues (Man₈₋₁₄GlcNAcGlcNAc), whereas the rest can be much bigger and contain >30 mannose residues (Man_{>30}GlcNAcGlcNAc). However, even the latter type is much smaller than the N-glycans found on proteins produced in S. cerevisiae (Man_{>50}GlcNAcGlcNAc). O-linked glycans on proteins produced in Pichia are much less well-studied. O-linked glycans with up to five mannose residues in the sugar chain have been described. All of these have been α1,2-linked and they may be phosphorylated.

Recently, a U.S.-based company named GlycoFi was formed in order to commercialize a number of Pichia pastoris strains that had been genetically modified to produce only one well-defined human form of N-linked glycans on proteins expressed in the specific strain. N-linked glycans are important for the parameters mentioned above. However, there have been no attempts in terms of trying to humanize O-glycans on proteins expressed in yeast. A number of biological functions, for example the adhesion of white blood cells to the vascular endothelium during inflammation, are mediated by O-glycans. Recombinant proteins with a defined, human-like O-glycan phenotype can therefore be expected to have a therapeutic value—a value that is mostly confined to the sugar chains themselves. Thus a need exists for a eukaryotic cell that can produce humanized O-linked glycans.

### SUMMARY OF THE INVENTION

The presence of N- and O-linked mannose on yeast produced glycoproteins can, if conjugated to a vaccine antigen, be utilized for specific targeting of the immune system with the aim of creating an enhanced immune response to antigens present on e.g. viruses, bacteria and cancer cells. This can be achieved due to the presence of mannose-binding receptors on certain cells of the human immune system. The mannose-binding receptors include the macrophage mannose receptor (MMR; CD206), which was the first discovered of a family of four mammalian endocytic receptors comprised of an extracellular region containing a cystein-rich (CR) domain, a domain containing fibronectin type two repeats (FNII) and multiple C-type lectin-like carbohydrate recognition domains (CTLD), a transmembrane domain and a short cytoplasmic tail. The family also includes the phospholipase A2 receptor, Endo180 and DEC205 (CD205), but only the MMR and Endo180 have the capacity to bind carbohydrates in a Ca²⁻-dependent manner. They are all type I proteins and contain multiple CTLDs. Another receptor binding high mannose structures is a type II protein on dendritic cells that was first described as a receptor interacting with intercellular adhesion molecule (ICAM)-3 and was therefore named dendritic cell-specific ICAM-3-grabbing nonintegrin (DC-SIGN; CD209). Both the MMR and DC-SIGN have the capacity to direct internalized antigens into endocytic pathways that result in MHC presentation and subsequent T cell activation. Antibodies specific for MMR or DC-SIGN have upon coupling to tumor-associated antigens been shown to stimulate both MHC class I and II-restricted T cell responses. Further, it was recently shown that ovalbumin (OVA) containing either O- or N-glycans, or both, when expressed in the yeast, Pichia pastoris, were more potent than the unmannosylated OVA at inducing OVA-specific CD4⁺ T cell proliferation.

However, for glycoproteins destined for other therapeutic uses than to enhance the immune response towards a specific antigen the nonhuman glycosylation phenotype characterized by oligosaccharides with a high number of mannose residues will trigger an unwanted immune response in humans, leading to a low therapeutic value.

Accordingly, the invention provides fusion proteins containing mannose residues that can be used as aduvants or vaccines. In addition, the invention also provides genetically engineered cells that express humanized glycoproteins.

In one aspect the invention provides a fusion polypeptide containing first polypeptide linked to a second polypeptide. The first polypeptide is mannosylated. By mannosylated is meant that the first polypeptide contains one or more mannose residues. For example, the two, three, four, five, six, seven, eight, nine, ten, fifteen, twenty or more mannose residues per glycan. Optionally, the first polypeptide is hypermannosylated. The mannose residues are N-linked or O-linked

The first polypeptide is a mucin polypeptide. Mucins include for example PSGL-1, MUC1, MUC2, MUC3a, MUC3b, MUC4, MUC5a, MUC5b, MUC5c, MUC6, MUC10, MUC11, MUC12, MUC13, MUC15, MUC16, MUC17, CD34, CD43, CD45, CD96, GlyCAM-1, MAdCAM, or a fragment thereof The polypeptide is a monomer. Alternatively, the polypeptide is a dimer. Preferably, polypeptide is for example a P-selectin glycoprotein ligand-1 polypeptide. The polypeptide includes at least a region of a P-selectin glycoprotein ligand-1, such as the extracellular portion of a P-selectin glycoprotein ligand-1. Alternatively, the first polypeptide is an alpha glycoprotein such as an alpha 1-acid glycoprotein (i.e., orosomuciod or AGP) or portion thereof.

The second polypeptide comprises at least a region of an immunoglobulin polypeptide. For example, the second polypeptide includes a region of a heavy chain immunoglobulin polypeptide, such as an F_{c} region or an F_{ab} region.

The mannosylated fusion polypetides of the invention can be formulated into adjuvant composition. The adjuvant composition can additionally contain a polypeptide carrying Gal1,2Gal epitopes.

Optionally, the mannosylated fusion polypeptide further contain an antigen The antigen is a for a example a virus, a bacteria or a fungus. For example, the antigen is Hepatitis C, HIV, Hepatitis B, Papilloma virus, Malaria, Tuberculosis, Herpes Simplex Virus, Chlamydia, or Influenza, or, a biological component thereof such as a peptide, protein, lipid carbohydrate, hormone or combination thereof. Alternatively, the antigen is a tumor associated antigen such as a breast, lung, colon, prostate, pancreatic, cervical or melanoma tumor-associated antigen. Optionally, the antigen is operably linked to the mannosylated fusion polypeptide. For example the antigen is covalently linked to the antigen. Alternatively, the is associated with the adjuvant polypeptide non-covalently.

The present invention further relates to an isolated nucleic acid encoding the fusion polypeptide, a vector including this isolated nucleic acid, and a cell comprising this vector. The vector further contains a nucleic acid encoding the antigen polypeptide. Preferably, the nucleic acid encoding the fusion polypeptide is expressed in a yeast cell. For example, the cell is Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei. In one embodiment, the invention provides a yeast cell comprising a nucleic acid construct encoding a P-selectin glycoprotein ligand-1 polypeptide or an alpha 1-acid glycoprotein of portion therof operably linked to at least a region of an immunoglobulin polypeptide, e.g. a heavy chain.

The invention also features a methods of immunization. A subject is immunized by administering to subject in need thereof a mannosylated fusion polypeptide according to the invention and an antigen. The antigen is covalently linked to the antigen. Alternatively, the is associated with the adjuvant polypeptide non-covalently. In a further aspect, the present invention includes a method of preventing or alleviating a symptom of cancer in a subject by identifying a subject in need suffering from or at risk of developing cancer and administering to the subject a mannosylated fusion polypeptide and a tumor associated antigen. according to the invention. For example the subject is suffering from or at risk of developing melanoma, breast, lung, colon, prostate, pancreatic, cervical cancer. A subject suffering from or at risk of developing cancer is identified by methods know in the art for the particular disorder.

In a further aspect, the invention provides cell lines having genetically modified glycosylation pathways that allow them to carry out a sequence of enzymatic reactions, which mimic the processing of O-linked glycoproteins in humans. Recombinant proteins expressed in these engineered hosts yield glycoproteins more similar, if not substantially identical, to their human counterparts. The lower eukaryotes, ordinarily produce O-glycans having at least five mannose residue. The cell is unicellular and multicellular fungi such as Pichia pastoris, Hansenulapolymorpha, Pichia stiptis, Pichia methanolica, Pichia sp., Kluyveromyces sp., Candida albicans, Aspergillus nidulans, and Trichoderma reseei, are modified to produce O-glycans or other structures along human glycosylation pathways. This is achieved using a combination of engineering and/or selection of strains which: do not express certain enzymes which create the undesirable complex structures characteristic of the fungal glycoproteins, which express exogenous enzymes selected either to have optimal activity under the conditions present in the fungi where activity is desired, or which are targeted to an organelle where optimal activity is achieved, and combinations thereof wherein the genetically engineered eukaryote expresses multiple exogenous enzymes required to produce “human-like” glycoproteins. Undesirable complex structures include high mannose structure. By hign mannose structure is meant eight or more mannose residues per oligosaccharide chain.

The cell is engineered to express one or more exogenous N-acetylgalactosaminyltransferase. Optionally, exogenous enzyme is targeted to the endoplasmic reticulum or Golgi apparatus of the cell.

Optionally, the glycosylation pathway of an eukaryotic microorganism is modified by (a) constructing a DNA library including at least two genes encoding exogenous glycosylation enzymes; (b) transforming the microorganism with the library to produce a genetically mixed population expressing at least two distinct exogenous glycosylation enzymes; (c) selecting from the population a microorganism having the desired glycosylation phenotype. In a preferred embodiment, the DNA library includes chimeric genes each encoding a protein localization sequence and a catalytic activity related to glycosylation. Organisms modified using the method are useful for producing glycoproteins having a glycosylation pattern similar or identical to mammals, especially humans.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a photograph of Western blot analysis of PSGL-1/mIgG2b fusion proteins produced in different clones of Pichia pastoris at 0, 24, 48 and 72 h of induction. The fusion proteins were analysed under non-reducing conditions on 4-12% bis-tris gels, electroblotted onto nitrocellulose membranes and stained with an HRP-conjugated goat anti-mIgG(Fc) antibody.

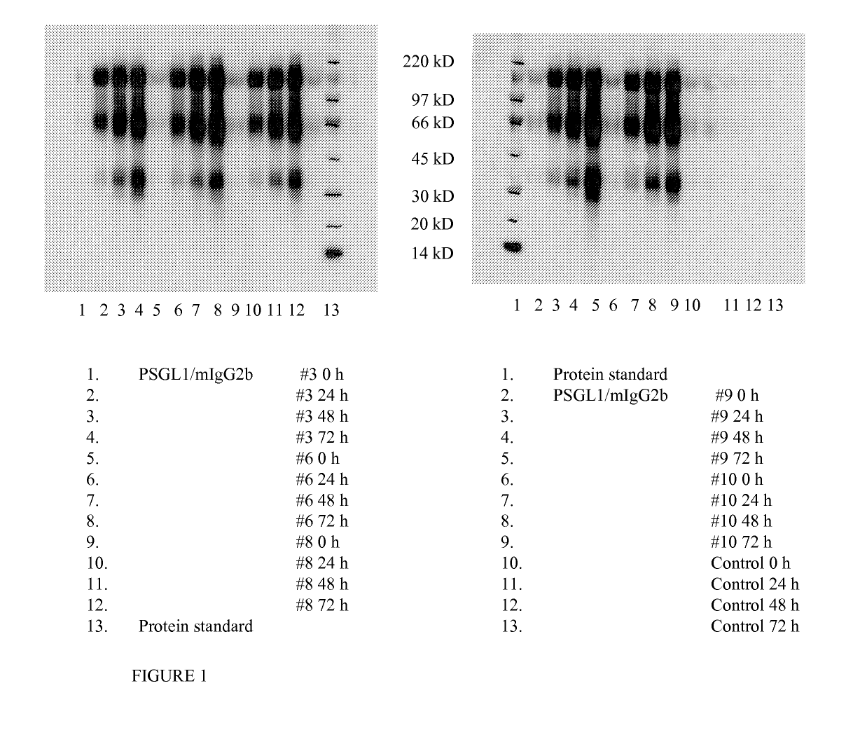

FIG. 2 is a photograph of Western blot analysis of PSGL-1/mIgG2b fusion proteins produced in different clones (1-5) of Pichia pastoris. The fusion proteins were analysed under non-reducing conditions on 4-12% bis-tris gels, electroblotted onto nitrocellulose membranes and stained with A) an HRP-conjugated goat anti-mIgG(Fc) antibody, and B) the lectin Concanavalin A which recognizes mannosylated glycan structures.

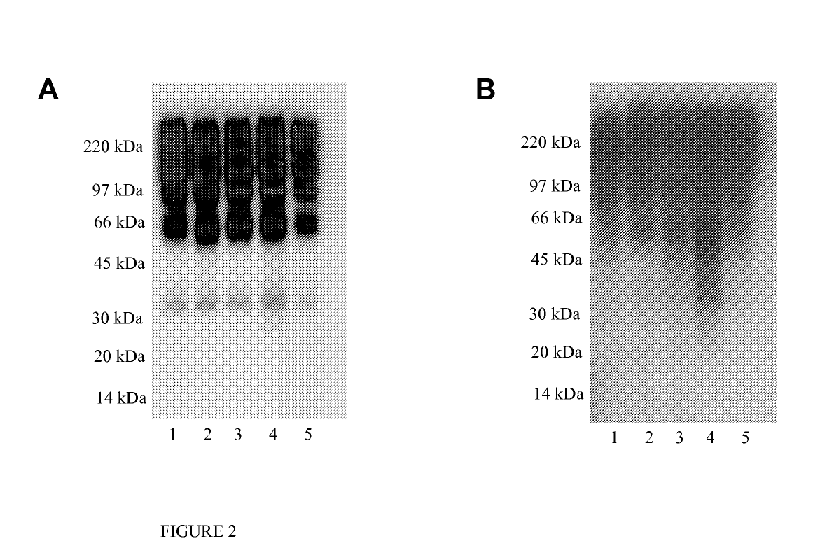

FIG. 3 is a photograph of Western blot analysis of AGP-1/mIgG2b fusion proteins (a, lysed cells; b, cell supernatant) produced in different clones (1-4) of Pichia pastoris. The fusion proteins were analysed under non-reducing conditions on 4-12% bis-tris gels, electroblotted onto nitrocellulose membranes and stained with A) an HRP-conjugated goat anti-mIgG(Fc) antibody, and B) an anti-AGP-1 antibody. C corresponds to PSGL-1/mIgG2b produced in CHO cells.

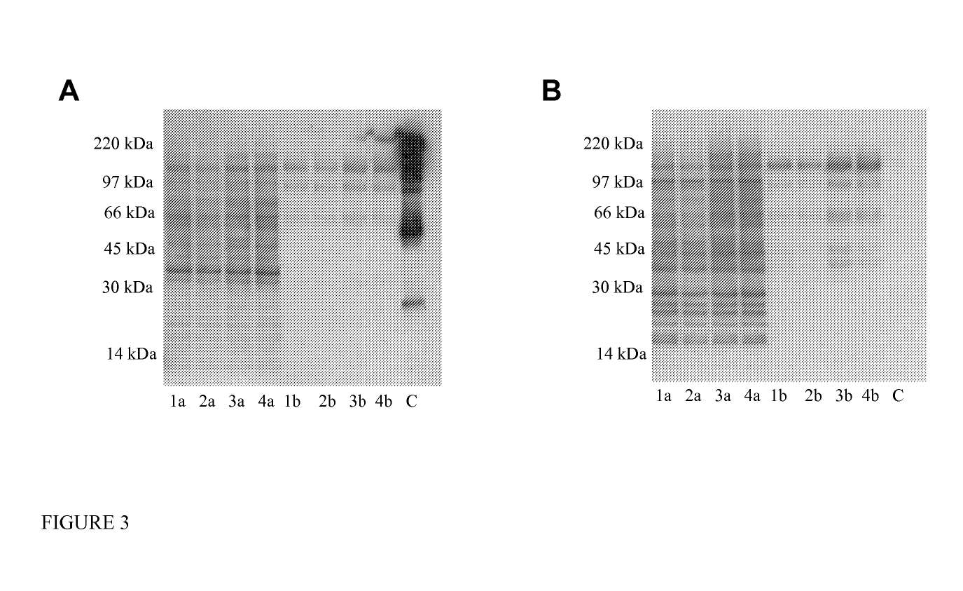

### DETAILED DESCRIPTION OF THE INVENTION

The methods and recombinant lower eukaryotic strains described herein are used to make “humanized glycoproteins”. The recombinant lower eukaryotes are made by engineering lower eukaryotes, which may not express one or more enzymes involved in production of high mannose structures, to express the enzymes required to produce human-like sugars. As used herein, a lower eukaryote is a unicellular or filamentous fungus. As used herein, a “humanized glycoprotein” refers to a protein having attached thereto O-glycans commonly expressed on human mucins and mucin-like proteins (see below), and the synthetic intermediates (which are also useful and can be manipulated further in vitro). This is achieved by cloning in different glycosyltransferases involved in production of O-glycans on human mucins or mucin-like proteins, i.e., enzymes selected to have optimal activity under the conditions present in the organisms at the site where proteins are glycosylated, or by targeting the enzymes to the organelles where activity is desired. In addition, some yeast endogenous mannosyltransferases may be knocked out or knocked down to avoid competition between inserted and endogenous glycosyltransferases. The invention also provides methods in which the high number of mannose residues expressed on glycoproteins produced in yeast are useful in targeting mannose receptors of the human immune system. Thus, in another aspect the invention also provides fusion proteins that are mannosylated, either N- or O-linked, or both.

O-linked glycans are usually attached to the peptide chain through serine or threonine residues. O-linked glycosylation is a true post-translational event and does not require an oligosaccharide precursor for protein transfer. The most common type of O-linked glycans contain an initial GalNAc residue (or Tn epitope), these are commonly referred to as mucin-type glycans. Other O-linked glycans include glucosamine, xylose, galactose, fucose, or mannose as the initial sugar bound to the Ser/Thr residues. O-linked glycoproteins are usually large proteins (>200 kDa) carrying O-glycans that are commonly bianttennary with comparatively less branching than N-glycans. Glycosylation generally occurs in high-density clusters and may contribute as much as 50-80% to the overall mass. O-linked glycans tend to be very heterogeneous, hence they are generally classified by their core structure. Nonelongated O-GlcNAc groups have been recently shown to be related to phosphorylation states and dynamic processing related to cell signaling events in the cell. O-linked glycans are prevalent in most secretory cells and tissues. They are present in high concentrations in the zona pelucida surrounding mammalian eggs and may funtion as sperm receptors (ZP3 glycoprotein). O-linked glycans are also involved in hematopoiesis, inflammation response mechanisms, and the formation of ABO blood antigens.

Elongation and termination of O-linked glycans is carried out by several glycosyltransferases. One notable core structure is the Galβ(1-3)GalNAc (core 1) sequence that has antigenic properties. Termination of O-linked glycans usually includes Gal, GlcNAc, GalNAc, Fuc, or sialic acid. By far the most common modification of the core Galβ(1-3)GalNAc is mono-, di-, or trisialylation. A less common, but widely distributed O-linked hexasaccharide structure contains β(1-4)-linked Gal and β(1-6)-linked GlcNAc as well as sialic acid.

Production of Humanized Glycoproteins

Preferably, eukaryotic strains which do not express one or more enzymes involved in the production of N-glycan high mannose structures are used to prevent immunogenic reactions towards possible N-glycans situated on the mucin or mucin-like model fusion protein. These strains can be engineered or be one of the many such mutants already described in yeasts, including a hypermannosylation-minus (OCH1) mutant in Pichia pastoris.

The strains can be engineered one enzyme at a time, or a library of genes encoding potentially useful enzymes can be created, and those strains having enzymes with optimal activities or producing the most “human-like” glycoproteins, selected.

Yeast and filamentous fungi have both been successfully used for the production of recombinant proteins, both intracellular and secreted (Cereghino, J. L. and J. M. Cregg 2000 FEMS Microbiology Reviews 24(1): 45 66; Harkki, A., et al. 1989 Bio-Technology 7(6): 596; Berka, R. M., et al. 1992 Abstr. Papers Amer. Chem. Soc. 203: 121-BIOT; Svetina, M., et al. 2000 J. Biotechnol. 76(2 3): 245 251).

Although glycosylation in yeast and fungi is very different than in humans, some common elements are shared. The first step of N-glycosylation, the transfer of the core oligosaccharide structure to the nascent protein, is highly conserved in all eukaryotes including yeast, fungi, plants and humans. Subsequent processing of the core oligosaccharide, however, differs significantly in yeast and involves the addition of several mannose sugars. This step is catalyzed by mannosyltransferases residing in the Golgi (e.g. OCH1, MNT1, MNN1, etc.), which sequentially add mannose sugars to the core oligosaccharide. The resulting structure is undesirable for the production of humanoid proteins and it is thus desirable to reduce or eliminate mannosyl transferase activity. Mutants of S. cerevisiae, deficient in mannosyl transferase activity (e.g. och1 or mnn9 mutants) have shown to be non-lethal and display a reduced mannose content in the oligosacharide of yeast glycoproteins. Other oligosacharide processing enzymes, such as mannosylphophate transferase may also have to be eliminated depending on the host's particular endogenous glycosylation pattern. After reducing undesired endogenous glycosylation reactions the formation of complex O-glycans is engineered into the host system. This requires the stable expression of several enzymes and sugar-nucleotide transporters. Moreover, one has to locate these enzymes in a fashion such that a sequential processing of the maturing glycosylation structure is ensured.

The methods described herein are useful for producing glycoproteins, especially glycoproteins used therapeutically in humans. Such therapeutic proteins are typically administered by injection, orally, pulmonary, or by other means.

The initial addition of a GalNAc to serine or threonine in the peptide sequence is performed by UDP-GalnAc-polypeptide N-acetylgalactosaminyltransferases (ppGalnAcTs). Fourteen ppGalNAcTs have been identified to date, ten of them in humans. The different ppGalNAcTs seem to be differently expressed in tissues, some overlapping and with a more ubiquitous expression than others. Further, individual ppGalNAcTs seem to have different peptide substrate specificities. ppGalNAcT1 is highly inhibited by neighboring glycosylated residues, while neighboring peptide residues seem to have minor influence on its activity, thus suggesting that ppGalNAcT1 is responsible for the initial glycosylation of peptides. The core 1 structure is generated by a β1,3-galactosyltransferase (C1 β3GalT). To days date, only one gene encoding a C1 β3GalT enzyme has been cloned. The C1 β3GalT is ubiquitously expressed in mammals and has been shown to require a chaperone for its activity. The core 2 structure is produced by the addition of a GlcNAc in a β1,6-linkage to core 1. Three core 2 N-acetylglucosaminyltransferases (C2 GnTs) have been cloned. C2 GnT-I has a widespread occurrence. In particular, it is highly expressed in spleen, which indicates a strong expression in B-cells. C2 GnT-II transcripts are highly expressed in mucin producing organs, such as the colon, small intestine, trachea, and stomach. This enzyme was shown to also have core 4 branching activity, which is not seen for C2 GnT-I. A third C2 GnT (C2 GnT-III) has been cloned that, like C2 GnT-I, have mainly core 2 branching activity. Northern blot analysis revealed the transcript of this enzyme to be highly expressed in thymus, while only low levels could be detected in other organs. Core 3 is synthesized by C3 GnT-VI, which adds a GlcNAc in a β1,3-linkage to the innermost GalNAc. Thus, this enzyme competes with the C1 β3GalT. The core 3 structure can then be elongated into type 4 by the addition of a GlcNAc in a β1,6-linkage to the peptide-linked GalNAc. The different core structures can be produced by expression of the above mentioned enzymes in yeast cells.

O-glycan terminal determinants vary even further on human glycoproteins. The majority of serum and membrane glycoproteins express mono- or disialylated core 1 structures. However, longer O-glycans terminating in e.g. blood group (ABH) and Lewis antigens can be found. Expecially, such structures are present on different cells of the hemopioetic lineage, e.g. sialyl Lewis x (SLe^{x}) on P-selectin glycoproteins ligand-1 (PSGL-1) expressed on leukocytes and interacting with P-selectin present on activated endothelial cells. Also, O-glycans may express α1,4-linked GlcNAc, a structure unique for this group of glycans. The terminal determinants are often expressed on lactosamine (LacNAc), or even branched repetitive LacNAc units (i and I antigens). Both branches of the trisaccharide cores (core 2 and 4) may be elongated, but the C6-branch is generally preferred over the C3-branch. The genes of the glycosyltransferases responsible for the production of above mentioned terminal determinants have been cloned and can therefore be inserted into yeast cells in order to promote the production of human-like O-glycans.

The method described herein may be used to engineer the glycosylation pattern of a wide range of lower eukaryotes (e.g. Hansenula polymorpha, Pichia stiptis, Pichia methanolica, Pichia sp, Kluyveromyces sp, Candida albicans, Aspergillus nidulans, Trichoderma reseei etc.). Pichia pastoris is used as an example. Similar to other lower eukaryotes, P. pastoris produces Man₉GlcNAc₂ structures in the ER. Glycoproteins produced in yeast cells modified as described above will express human-like O-glycans. However, the chosen proteins may also contain one or more N-glycosylation sites. In order to avoid the expression of high-mannose N-glycans on the produced glycoproteins it is of importance to eliminate the ability of the fungus to hypermannosylate existing Man₉GlcNAc₂ structures. This can be achieved by either selecting for a fungus that does not hypermannosylate, or by genetically engineering such a fungus.

Genes that are involved in this process have been identified in Pichia pastoris and by creating mutations in these genes one is able to reduce the production of “undesirable” glycoforms. Such genes can be identified by homology to existing mannosyltransferases (e.g. OCH1, MNN4, MNN6, MNN1), found in other lower eukaryotes such as C. albicans, Pichia angusta or S. cerevisiae or by mutagenizing the host strain and selecting for a phenotype with eliminated or reduced mannosylation. Alternatively, one may be able to complement particular phenotypes in related organisms. For example, in order to obtain the gene or genes encoding 1,6-mannosyltransferase activity in P. pastoris, one would carry out the following steps. OCH1 mutants of S. cerevisiae are temperature sensitive and are slow growers at elevated temperatures. One can thus identify functional homologues of OCH1 in P. pastoris by complementing an OCH1 mutant of S. cerevisiae with a P. pastoris DNA or cDNA library. Such mutants of S. cerevisiae may be found e.g., see the Saccharomyces genome link at the Stanford University website and are commercially available. Mutants that display a normal growth phenotype at elevated temperature, after having been transformed with a P. pastoris DNA library, are likely to carry an OCH1 homologue of P. pastoris. Such a library can be created by partially digesting chromosomal DNA of P. pastoris with a suitable restriction enzyme and after inactivating the restriction enzyme ligating the digested DNA into a suitable vector, which has been digested with a compatible restriction enzyme. Suitable vectors are pRS314, a low copy (CEN6/ARS4) plasmid based on pBluescript containing the Trpl marker (Sikorski, R. S., and Hieter, P., 1989, Genetics 122, pg 19 27) or pFL44S, a high copy (2.beta.) plasmid based on a modified pUC19 containing the URA3 marker (Bonneaud, N., et al., 1991, Yeast 7, pg. 609 615). Such vectors are commonly used by academic researchers or similar vectors are available from a number of different vendors such as Invitrogen (Carlsbad, Calif.), Pharmacia (Piscataway, N.J.), New England Biolabs (Beverly, Mass.). Examples are pYES/GS, 2.beta. origin of replication based yeast expression plasmid from Invitrogen, or Yep24 cloning vehicle from New England Biolabs. After ligation of the chromosomal DNA and the vector one may transform the DNA library into strain of S. cerevisiae with a specific mutation and select for the correction of the corresponding phenotype. After sub-cloning and sequencing the DNA fragment that is able to restore the wild-type phenotype, one may use this fragment to eliminate the activity of the gene product encoded by OCHi in P. pastoris.

Alternatively, if the entire genomic sequence of a particular fungus of interest is known, one may identify such genes simply by searching publicly available DNA databases, which are available from several sources such as NCBI, Swissprot etc. For example by searching a given genomic sequence or data base with a known 1,6 mannosyltransferase gene (OCH1) from S. cerevisiae, one can able to identify genes of high homology in such a genome, which a high degree of certainty encodes a gene that has 1,6 mannosyltransferase activity. Homologues to several known mannosyltransferases from S. cerevisiae in P. pastoris have been identified using either one of these approaches. These genes have similar functions to genes involved in the mannosylation of proteins in S. cerevisiae and thus their deletion may be used to manipulate the glycosylation pattern in P. pastoris or any other fungus with similar glycosylation pathways.

The creation of gene knock-outs, once a given target gene sequence has been determined, is a well-established technique in the yeast and fungal molecular biology community, and can be carried out by anyone of ordinary skill in the art (R. Rothsteins, (1991) Methods in Enzymology, vol. 194, p. 281). In fact, the choice of a host organism may be influenced by the availability of good transformation and gene disruption techniques for such a host. If several mannosyltransferases have to be knocked out, the method developed by Alani and Kleckner allows for the repeated use of the URA3 markers to sequentially eliminate all undesirable endogenous mannosyltransferase activity. This technique has been refined by others but basically involves the use of two repeated DNA sequences, flanking a counter selectable marker. For example: URA3 may be used as a marker to ensure the selection of a transformants that have integrated a construct. By flanking the URA3 marker with direct repeats one may first select for transformants that have integrated the construct and have thus disrupted the target gene. After isolation of the transformants, and their characterization, one may counter select in a second round for those that are resistant to 5′FOA. Colonies that able to survive on plates containing 5′FOA have lost the URA3 marker again through a crossover event involving the repeats mentioned earlier. This approach thus allows for the repeated use of the same marker and facilitates the disruption of multiple genes without requiring additional markers.

Eliminating specific mannosyltransferases, such as 1,6 mannosyltransferase (OCH1), mannosylphosphate transferases (MNN4, MNN6, or genes complementing lbd mutants) in P. pastoris, allows for the creation of engineered strains of this organism which synthesize primarily Man₈GlcNAc₂ and thus can be used to further modify the glycosylation pattern to more closely resemble more complex human glycoform structures. A preferred embodiment of this method utilizes known DNA sequences, encoding known biochemical glycosylation activities to eliminate similar or identical biochemical functions in P. pastoris, such that the glycosylation structure of the resulting genetically altered P. pastoris strain is modified.

Most enzymes that are active in the ER and Golgi apparatus of S. cerevisiae have pH optima that are between 6.5 and 7.5. All previous approaches to reduce mannosylation by the action of recombinant mannosidases have concentrated on enzymes that have a pH optimum around pH 5.0 (Martinet et al., 1998, and Chiba et al., 1998), even though the activity of these enzymes is reduced to less than 10% at pH 7.0 and thus most likely provide insufficient activity at their point of use, the ER and early Golgi of P. pastoris and S. cerevisiae. A preferred process utilizes an α-mannosidase in vivo, where the pH optimum of the mannosidase is within 1.4 pH units of the average pH optimum of other representative marker enzymes localized in the same organelle(s). The pH optimum of the enzyme to be targeted to a specific organelle should be matched with the pH optimum of other enzymes found in the same organelle, such that the maximum activity per unit enzyme is obtained.

When one attempts to trim high mannose structures to yield Man₅GlcNAc₂ in the ER or the Golgi apparatus of S. cerevisiae, one may choose any enzyme or combination of enzymes that (1) has/have a sufficiently close pH optimum (i.e. between pH 5.2 and pH 7.8), and (2) is/are known to generate, alone or in concert, the specific isomeric Man₅GlcNAc₂ structure required to accept subsequent addition of GlcNAc by GnT I. Any enzyme or combination of enzymes that has/have shown to generate a structure that can be converted to Man₅GlcNAc₂ by GnT I in vitro would constitute an appropriate choice. This knowledge may be obtained from the scientific literature or experimentally by determining that a potential mannosidase can convert Man₈GlcNAc₂ to Man₅GlcNAc₂-PA and then testing, if the obtained Man₅GlcNAc₂-PA structure can serve a substrate for GnT I and UDP-GlcNAc to give GlcNAcMan.sub.5GlcNAc.sub.2 in vitro. For example, mannosidase IA from a human or murine source would be an appropriate choice.

Previous approaches to reduce mannosylation by the action of cloned exogenous mannosidases have failed to yield glycoproteins having a sufficient fraction (e.g. >27 mole %) of O-glycans (Martinet et al., 1998, and Chiba et al., 1998). These enzymes should function efficiently in ER or Golgi apparatus to be effective in converting nascent glycoproteins.

A second step of the process involves the sequential addition of sugars to the nascent carbohydrate structure by engineering the expression of glucosyltransferases into the Golgi apparatus. This process first requires the functional expression of GnT I in the early or medial Golgi apparatus as well as ensuring the sufficient supply of UDP-N-acetyl-D-galactosaminide.

Since the ultimate goal of this genetic engineering effort is a robust protein production strain that is able to perform well in an industrial fermentation process, the integration of multiple genes into the fungal chromosome involves careful planing. The engineered strain are transformed with a range of different genes, and these genes will have to be transformed in a stable fashion to ensure that the desired activity is maintained throughout the fermentation process. Any combination of the following enzyme activities will have to be engineered into the fungal protein expression host: sialyltransferases, mannosidases, fucosyltransferases, galactosyltransferases, glucosyltransferases, GlcNAc transferases, ER and Golgi specific transporters (e.g. syn and antiport transporters for UDP-galactose and other precursors), other enzymes involved in the processing of oligosaccharides, and enzymes involved in the synthesis of activated oligosaccharide precursors such as UDP-galactose, CMP-N-acetylneuraminic acid. At the same time a number of genes which encode enzymes known to be characteristic of non-human glycosylation reactions, will have to be deleted.

Glycosyltransferases and mannosidases line the inner (luminal) surface of the ER and Golgi apparatus and thereby provide a “catalytic” surface that allows for the sequential processing of glycoproteins as they proceed through the ER and Golgi network. In fact the multiple compartments of the cis, medial, and trans Golgi and the trans-Golgi Network (TGN), provide the different localities in which the ordered sequence of glycosylation reactions can take place. As a glycoprotein proceeds from synthesis in the ER to full maturation in the late Golgi or TGN, it is sequentially exposed to different glycosidases, mannosidases and glycosyltransferases such that a specific carbohydrate structure may be synthesized. Much work has been dedicated to revealing the exact mechanism by which these enzymes are retained and anchored to their respective organelle. The evolving picture is complex but evidence suggests that stem region, membrane spanning region and cytoplasmic tail individually or in concert direct enzymes to the membrane of individual organelles and thereby localize the associated catalytic domain to that locus.

Targeting sequences are well known and described in the scientific literature and public databases, as discussed in more detail below with respect to libraries for selection of targeting sequences and targeted enzymes.

Mannosylated Fusion Proteins

Also included in the invention are fusion proteins carrying N- or O-linked, or both, oligomannose structures. The fusion proteins of the invention are useful in enhancing the response towards specific antigens. This can be achieved by conjugation of the mannosylated fusion protein to vaccine antigens. The fusion proteins will target the vaccine antigen to macrophages and dendritic cells via binding to mannose-binding receptors, thereby increasing the immunogenicity of various vaccine constituents. Accordingly, the mannosylated fusion proteins of the invention are useful as vaccine adjuvants. Such targeting is also useful for various imaging applications.

The mannose-binding receptors include the macrophage mannose receptor (MMR; CD206), which was the first discovered of a family of four mammalian endocytic receptors comprised of an extracellular region containing a cystein-rich (CR) domain, a domain containing fibronectin type two repeats (FNII) and multiple C-type lectin-like carbohydrate recognition domains (CTLD), a transmembrane domain and a short cytoplasmic tail. The family also include the phospholipase A2 receptor, Endo180 and DEC205 (CD205), but only the MMR and Endo180 have the capacity to bind carbohydrates in a Ca²⁺-dependent manner. They are all type I proteins and contain multiple CTLDs. Another receptor binding high mannose structures is a type II protein on dendritic cells that was first described as a receptor interacting with intercellular adhesion molecule (ICAM)-3 and was therefore named dendritic cell-specific ICAM-3-grabbing nonintegrin (DC-SIGN; CD209). Both the MMR and DC-SIGN have the capacity to direct internalized antigens into endocytic pathways that result in MHC presentation and subsequent T cell activation. Antibodies specific for MMR or DC-SIGN have upon coupling to tumor-associated antigens been shown to stimulate both MHC class I and II-restricted T cell responses. Further, it was recently shown that ovalbumin (OVA) containing either O- or N-glycans, or both, when expressed in the yeast, Pichia pastoris, were more potent than the unmannosylated OVA at inducing OVA-specific CD4⁺ T cell proliferation.

The invention provides glycoprotein-immunoglobulin fusion proteins (refered to herein as “Man fusion protein or Man fusion peptides”) containing multiple mannose epitopes.

The Man fusion proteins or Man fusion peptides are more efficient on a carbohydrate molar basis in inhibiting mannose receptor-ligand binding as compared to free saccharrides. The reason for this is most likely the multivalent presentation of the mannosylated glycans as compared to monovalent free oligosaccharides.

The mannosylated fusion peptide inhibits 2, 4, 10, 20, 50, 80, 100 or more-fold greater number of mannose receptor-ligand binding to an equivalent amount of free saccharrides.

In various aspects the invention provides fusion proteins that include a first polypeptide containing at least a portion of a glycoprotein, e.g. a mucin polypeptide or an alpha-globulin polypeptide, operatively linked to a second polypeptide. As used herein, a “fusion protein” or “chimeric protein” includes at least a portion of a glycoprotein polypeptide operatively linked to a non-mucin polypeptide.

A “mucin polypeptide” refers to a polypeptide having a mucin domain. The mucin polypeptide has one, two, three, five, ten, twenty or more mucin domains. The mucin polypeptide is any glycoprotein characterized by repetitive amino acid sequences, called tandem repeats, substituted with O-glycans. For example, a mucin polypeptide has every second or third amino acid being a serine or threonine. The mucin polypeptide is a secreted protein. Alternatively, the mucin polypeptide is a cell surface protein.

Mucin domains are rich in the amino acids threonine, serine and proline, where the oligosaccharides are linked via N-acetylgalactosamine to the hydroxy amino acids (O-glycans). A mucin domain comprises or alternatively consists of an O-linked glycosylation site. A mucin domain has 1, 2, 3, 5, 10, 20, 50, 100 or more O-linked glycosylation sites. A mucin polypeptide has 50%, 60%, 80%, 90%, 95% or 100% of its mass due to the glycan. A mucin polypeptide is any polypeptide encoded for by a MUC gene (i.e., MUC1, MUC2, MUC3a, MUC3b, MUC4, MUC5a, MUC5b, MUC5c, MUC6, MUC10, MUC11, MUC12, MUC13, MUC15, MUC16, MUC17). Alternatively, a mucin polypeptide is P-selectin glycoprotein ligand 1 (PSGL-1), CD34, CD43, CD45, CD96, GlyCAM-1, MAdCAM, or red blood cell glycophorins. Preferably, the mucin is PSGL-1.

An “alpha-globulin polypeptide” refers to a serum glycoprotein. Alpha-globulins include for example, enzymes produced by the lungs and liver, and haptoglobin, which binds hemoglobin together. An alpha-globulin is an alpha₁ or an alpha₂ globulin. Alpha₁ globulin is predominantly alpha₁antitrypsin, an enzyme produced by the lungs and liver. Alpha₂ globulin, which includes serum haptoglobin, is a protein that binds hemoglobin to prevent its excretion by the kidneys. Other alphaglobulins are produced as a result of inflammation, tissue damage, autoimmune diseases, or certain cancers. Preferably, the alpha-globulin is alpha-1-acid glycoprotein (i.e., orosomucoid).

A “non-mucin polypeptide” refers to a polypeptide of which at least less than 40% of its mass is due to glycans. As used herein, the following definitions are supplied in order to facilitate the understanding of this case. To the extent that the definitions vary from meanings known to those skilled in the art, the definitions below control.

By “biological component” is meant any compound created by or associated with a cell, tissue, bacteria, virus, or other biological entity, including peptides, proteins, lipids, carbohydrates, hormones, or combinations thereof.

By “adjuvant compound” is meant any compound that increases an immunogenic response or the immunogenicity of an antigen or vaccine.

By “antigen” is meant any compound capable of inducing an immunogenic response.

By “immunoglobulin” is meant any polypeptide or protein complex that is secreted by plasma cells and that functions as an antibody in the immune response by binding with a specific antigen. Immunoglobulins as used herein include IgA, IgD, IgE, IgG, and IgM. Regions of immunoglobulins include the Fc region and the Fab region, as well as the heavy chain or light chain immunoglobulins.

By “antigen presentation” is meant the expression of an antigen on the surface of a cell in association with one or more major hisocompatability complex class I or class II molecules. Antigen presentation is measured by methods known in the art. For example, antigen presentation is measured using an in vitro cellular assay as described in Gillis, et al., J. Immunol. 120: 2027 1978.

By “immunogenicity” is meant the ability of a substance to stimulate an immune response. Immunogenicity is measured, for example, by determining the presence of antibodies specific for the substance. The presence of antibodies is detected by methods know in the art, for example, an ELISA assay.

By “immune response” or “immunogenic response” is meant a cellular activity induced by an antigen, such as production of antibodies or presentation of antigens or antigen fragments.

By “proteolytic degradation” is meant degradation of the polypeptide by hydrolysis of the peptide bonds. No particular length is implied by the term “peptide.” Proteolytic degradation is measured, for example, using gel electrophoresis.

The “cell” includes any cell capable of antigen presentation. For example, the cell is a somatic cell, a B-cell, a macrophage or a dendritic cell.

Within a Man fusion protein of the invention the mucin polypeptide corresponds to all or a portion of a mucin or mucin-type protein. A Man fusion protein comprises at least a portion of a mucin or mucin-type protein. “At least a portion” is meant that the mucin polypeptide contains at least one mucin domain (e.g., an O-linked glycosylation site). The mucin protein comprises the extracellular portion of the polypeptide. For example, the mucin polypeptide comprises the extracellular portion of PSGL-b 1.

The alpha globulin polypeptide can corresponds to all or a portion of a alpha globulin polypeptide. A Man fusion protein comprises at least a portion of a alpha globulin polypeptide “At least a portion” is meant that the alpha globulin polypeptide contains at least one N-linked glycosylation site.

The first polypeptide is glycosylated by one or more glycotransferases. The first polypeptide is glycosylated by 2, 3, 4, 5 or more glycotransferases. Glycosylation is sequential or consecutive. Alternatively glycosylation is concurrent or random. By glycosyltransferases are referred to glycosyltransferases known to be involved in the production of N- or O-linked glycan chains, both mannosylated structures and human-like glycans. The first polypeptide contains greater that 40%, 50%, 60%, 70%, 80%, 90% or 95% of its mass due to carbohydrate

Within the fusion protein, the term “operatively linked” is intended to indicate that the first and second polypeptides are chemically linked (most typically via a covalent bond such as a peptide bond) in a manner that allows for O-linked and/or N-linked glycosylation of the first polypeptide. When used to refer to nucleic acids encoding a fusion polypeptide, the term operatively linked means that a nucleic acid encoding the mucin/mucin-type or alpha globulin polypeptide and the non-mucin polypeptide are fused in-frame to each other. The non-mucin polypeptide can be fused to the N-terminus or C-terminus of the mucin/mucin-type or alpha globulin polypeptide.

The Man fusion protein is linked to one or more additional moieties. For example, the Man fusion protein may additionally be linked to a GST fusion protein in which the Man fusion protein sequences are fused to the C-terminus of the GST (i.e., glutathione S-transferase) sequences. Such fusion proteins can facilitate the purification of the Man fusion protein. Alternatively, the Man fusion protein may additionally be linked to a solid support. Various solid supports are known to those skilled in the art. Such compositions can facilitate removal of anti-blood group antibodies. For example, the Man fusion protein is linked to a particle made of, e.g., metal compounds, silica, latex, polymeric material; a microtiter plate; nitrocellulose, or nylon or a combination thereof. The Man fusion proteins linked to a solid support are used as an absorber to remove microbes, bacterial toxins or other Man-binding proteins from biological sample, such as gastric tissue, blood or plasma.

Optionally, the Man fusion protein is linked to an antigen to form a vaccine. An “antigen” includes any compound to which an immune response is desired. An antigen includes any substance that, when introduced into the body, stimulates an immune response, such as the production of an antibody from a B cell, activation and expansion of T cells, and cytokine expression (e.g., interleukins). By a “B cell” or “B lymphocyte” is meant an immune cell that, when activated, is responsible for the production of antibodies. By a “T cell” or “T lymphocyte” is meant a member of a class of lymphocytes, further defined as cytotoxic T cells and helper T cells. T cells regulate and coordinate the overall immune response, identifying the epitopes that mark the antigens, and attacking and destroying the diseased cells they recognize as foreign. Antigens include for example, toxins, bacteria, foreign blood cells, and the cells of transplanted organs. Preferably, the antigen is Hepatitis C, HIV, Hepatitis B, Papilloma virus, Malaria, Tuberculosis, Herpes Simplex Virus, Chlamydia, and Influenza, or a biological component thereof, for example, a viral or bacterial polypeptide. In embodiments of the invention the adjuvant polypeptide is covalently linked to the antigen. For example, the Man fusion protein is linked to the antigen via a covalent bond such as a peptide bond. The antigen is fused to the N-terminus or C-terminus of the mucin polypeptide. Alternatively, the antigen is fused to an internal amino acid of the mucin polypeptide. By “internal amino acid” is meant an amino acid that is not at the N-terminal or C-terminal of a polypeptide. Similarly, the antigen is operably linked to the second polypeptide of the adjuvant polypeptide, most typically via a covalent bond such as a peptide bond. The antigen is fused to the N-terminus or C-terminus of the second polypeptide of the adjuvant polypeptide. Alternatively, the antigen is fused to an internal amino acid of the second polypeptide of the adjuvant polypeptide.

The Man fusion proteins includes a heterologous signal sequence (i.e., a polypeptide sequence that is not present in a polypeptide encoded by a mucin or a globulin nucleic acid) at its N-terminus. For example, the native mucin or alpha-glycoprotein signal sequence can be removed and replaced with a signal sequence from another protein. In certain host cells (e.g., mammalian host cells), expression and/or secretion of polypeptide can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene is synthesized by conventional techniques including automated DNA synthesizers. Alternatively, PCR amplification of gene fragments is carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A mucin or a alpha-globulin encoding nucleic acid can be cloned into such an expression vector such that the fusion moiety is linked in-frame to the immunoglobulin protein.

Man fusion polypeptides may exist as oligomers, such as dimers, trimers or pentamers. Preferably, the Man fusion polypeptide is a dimer.

The first polypeptide, and/or nucleic acids encoding the first polypeptide, is constructed using mucin/mucin-type or alpha-globulin encoding sequences known in the art. Suitable sources for mucin polypeptides and nucleic acids encoding mucin polypeptides include GenBank Accession Nos. NP663625 and NM145650, CAD10625 and AJ417815, XP140694 and XM140694, XP006867 and XM006867 and NP00331777 and NM009151 respectively, and are incorporated herein by reference in their entirety. Suitable sources for alpha-globulin polypeptides and nucleic acids encoding alpha-globulin polypeptides include GenBank Accession Nos. AAH26238 and BC026238; NP000598; and BC012725, AAH12725 and BC012725, and NP44570 and NM053288 respectively, and are incorporated herein by reference in their entirety.

The mucin polypeptide moiety is provided as a variant mucin polypeptide having a mutation in the naturally-occurring mucin sequence (wild type) that results in increased carbohydrate content (relative to the non-mutated sequence). For example, the variant mucin polypeptide comprised additional O-linked glycosylation sites compared to the wild-type mucin. Alternatively, the variant mucin polypeptide comprises an amino acid sequence mutations that results in an increased number of serine, threonine or proline residues as compared to a wild type mucin polypeptide. This increased carbohydrate content can be assessed by determining the protein to carbohydrate ratio of the mucin by methods known to those skilled in the art.

Similarly, the alpha-globulin polypeptide moiety is provided as a variant alpha-globulin polypeptide having a mutation in the naturally-occurring alpha-globulin sequence (wild type) that results in increased carbohydrate content (relative to the non-mutated sequence). For example, the variant alpha-globulin polypeptide comprised additional N-linked glycosylation sites compared to the wild-type alpha-globulin.

Alternatively, the mucin or alpha-globulin polypeptide moiety is provided as a variant mucin or alpha-globulin polypeptide having mutations in the naturally-occurring mucin or alpha-globulin sequence (wild type) that results in a mucin or alpha-globulin sequence more resistant to proteolysis (relative to the non-mutated sequence).

The first polypeptide includes full-length PSGL-1. Alternatively, the first polypeptide comprise less than full-length PSGL-1 polypeptide such as the extracellular portion of PSGL-1. For example the first polypeptide less than 400 amino acids in length, e.g. less than or equal to 300, 250, 150, 100, 50, or 25 amino acids in length.

The first polypeptide includes full-length alpha acid-globulin. Alternatively, the first polypeptide comprises less than full-length alpha acid globulin polypeptides. For example the first polypeptide less than 200 amino acids in length, e.g., less than or equal to 150, 100, 50, or 25 amino acids in length.

The second polypeptide is preferably soluble. In some embodiments, the second polypeptide includes a sequence that facilitates association of the Man fusion polypeptide with a second mucin or alpha globulin polypeptide. The second polypeptide includes at least a region of an immunoglobulin polypeptide. “At least a region” is meant to include any portion of an immunoglobulin molecule, such as the light chain, heavy chain, Fc region, Fab region, Fv region or any fragment thereof. Immunoglobulin fusion polypeptide are known in the art and are described in e.g., U.S. Pat. Nos. 5,516,964; 5,225,538; 5,428,130; 5,514,582; 5,714,147; and 5,455,165.

The second polypeptide comprises a full-length immunoglobulin polypeptide. Alternatively, the second polypeptide comprise less than full-length immunoglobulin polypeptide, e.g., a heavy chain, light chain, Fab, Fab₂, Fv, or Fc. Preferably, the second polypeptide includes the heavy chain of an immunoglobulin polypeptide. More preferably the second polypeptide includes the Fc region of an immunoglobulin polypeptide.

The second polypeptide has less effector function that the effector function of a Fc region of a wild-type immunoglobulin heavy chain. Alternatively, the second polypeptide has similar or greater effector function of a Fc region of a wild-type immunoglobulin heavy chain. An Fc effector function includes for example, Fc receptor binding, complement fixation and T cell depleting activity. (see for example, U.S. Pat. No. 6,136,310) Methods of assaying T cell depleting activity, Fc effector function, and antibody stability are known in the art. In one embodiment the second polypeptide has low or no affinity for the Fc receptor. Alternatively, the second polypeptide has low or no affinity for complement protein C1q.

Another aspect of the invention pertains to vectors, preferably expression vectors, containing a nucleic acid encoding mucin polypeptides, or derivatives, fragments, analogs or homologs thereof. The vector contains a nucleic acid encoding a mucin or alpha globulin polypeptide operably linked to an nucleic acid encoding an immunoglobulin polypeptide, or derivatives, fragments analogs or homologs thereof. Additionally, the vector comprises a nucleic acid encoding a glycotransferase. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., Man fusion polypeptides, mutant forms of Man fusion polypeptides, etc.).

The recombinant expression vectors of the invention can be designed for expression of Man fusion polypeptides in prokaryotic or eukaryotic cells. Preferably the Man fusion proteins are expressed in eukatyotic cells. Most preferably, the Man-fusion proteins are expressed in a yeast cell such as Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei.

The Man fusion polypeptide expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, Man fusion polypeptides can be expressed in bacterial cells such as E. coli, insect cells, yeast or mammalian cells (such as human, Chinese hamster ovary cells (CHO) or COS cells). Other suitable host cells are known to those skilled in the art. Preferably, the host cell is yeast.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the fusion polypeptides or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) Man fusion polypeptides. Accordingly, the invention further provides methods for producing Man fusion polypeptides using the host cells of the invention. In one embodiment, the method comprises culturing the host cell of invention (into which a recombinant expression vector encoding Man fusion polypeptides has been introduced) in a suitable medium such that Man fusion polypeptides is produced. In another embodiment, the method further comprises isolating Man polypeptide from the medium or the host cell.

The Man fusion polypeptides may be isolated and purified in accordance with conventional conditions, such as extraction, precipitation, chromatography, affinity chromatography, electrophoresis or the like. For example, the immunoglobulin fusion proteins may be purified by passing a solution through a column which contains immobilized protein A or protein G which selectively binds the Fc portion of the fusion protein. See, for example, Reis, K. J., et al., J. Immunol. 132:3098-3102 (1984); PCT Application, Publication No. WO87/00329. The fusion polypeptide may the be eluted by treatment with a chaotropic salt or by elution with aqueous acetic acid (1 M).

Alternatively, a Man fusion polypeptides according to the invention can be chemically synthesized using methods known in the art. Chemical synthesis of polypeptides is described in, e.g., A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The polypeptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of polypeptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action. See, e.g., Doherty, et al., 1993. J. Med. Chem. 36: 2585-2594; Kirby, et al., 1993. J. Med. Chem. 36:3802-3808; Morita, et al., 1994. FEBS Lett. 353: 84-88; Wang, et al., 1993. Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the polypeptide backbone. This strategy can be used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus [e.g., with K₃Fe(CN)₆ at pH 8.5] (Samson et al., Endocrinology, 137: 5182-5185 (1996)), or between two amino acid side chains. See, e.g., DeGrado, Adv Protein Chem, 39: 51-124 (1988). Disulfide bridges are also introduced into linear sequences to reduce their flexibility. See, e.g., Rose, et al., Adv Protein Chem, 37: 1-109 (1985); Mosberg et al., Biochem Biophys Res Commun, 106: 505-512 (1982). Furthermore, the replacement of cysteine residues with penicillamine (Pen, 3-mercapto-(D) valine) has been used to increase the selectivity of some opioid-receptor interactions. Lipkowski and Carr, Peptides: Synthesis, Structures, and Applications, Gutte, ed., Academic Press pp. 287-320 (1995).

Methods of Immunization

The Man-fusion proteins of the invention are also useful as vaccine adjuvant. The vaccines of the present invention have superior immunoprotective and immunotherapeutic properties over other vaccine lacking adjuvant polypeptides. Mucin-Ig fusion protein-containing vaccines have enhanced immunogenicity, safety, tolerability and efficacy. For example, the enhanced immunogenicity of the vaccine of the present invention may be greater than comparative non-adjuvant polypeptide-containing vaccines by 1.5-fold, 2-fold, 3-fold, 5-fold, 10-fold, 20-fold, 50-fold, 100-fold or more, as measured by stimuation of an immune response such as antibody production and/or secretion, activation and expansion of T cells, and cytokine expression (e.g., production of interleukins).

The cell surface of cancer cells often contains specific carbohydrates, polypeptides and other potential antibody epitopes that are not presence on the surface of non-cancerous cells. This antigen disparity allows the body's immune system to detect and respond to cancer cells. Mucin polypeptides have been associated with numerous cancers. For example, PSGL-1 has been associated with cancers, including lung cancer and acute myeloid leukemia (See Kappelmayer et al., Br J Haematol. 2001, 115(4):903-9). Also, MUC1-specific antibodies have been detected in sera from breast, pancreatic and colon cancer patients. It is clear that mucins can be recognized by the human immune system; therefore, immunity against tumor cells expressing specific antigens will be induced by vaccines containing mucin-Ig fusion proteins and a tumor cell-specific antigen. Immunity to tumor cells is measured by the extent of decrease of tumor size, decreased tumor vascularization, increased subject survival, or increased tumor cell apoptosis.

The invention provides a method of immunization of a subject. A subject is immunized by administration to the subject the vaccine including an adjuvant polypeptide, e.g. an Man fusion protein and an antigen. The subject is at risk of developing or suffering from an infection, e.g., bacterial, viral or fungal. Infections include, Hepatitis C, HIV, Hepatitis B, Papilloma virus, Malaria, Tuberculosis, Herpes Simplex Virus, Chlamydia, or Influenza. Alternatively, the subject is at risk of developing or suffering from cancer. The cancer is for example breast, lung, colon, prostate, pancreatic, cervical cancer or melanoma.

The methods described herein lead to a reduction in the severity or the alleviation of one or more symptoms of a infection or cancer. Infection and cancers diagnosed and or monitored, typically by a physician using standard methodologies A subject requiring immunization is identified by methods know in the art. For example subjects are immunized as outlined in the CDC's General Recommendation on Immunization (51(RR02) pp 1-36). Cancer is diagnosed for example by physical exam, biopsy, blood test, or x-ray.

The subject is e.g. any mammal, e.g., a human, a primate, mouse, rat, dog, cat, cow, horse, pig. The treatment is administered prior to diagnosis of the disorder. Alternatively, treatment is administered after diagnosis.

Efficaciousness of treatment is determined in association with any known method for diagnosing or treating the particular disorder. Alleviation of one or more symptoms of the disorder indicates that the compound confers a clinical benefit. By “efficacious” is meant that the treatment leads to decrease in size, prevalence, or metastatic potential of the cancer in a subject. When treatment is applied prophylactically, “efficacious” means that the treatment retards or prevents a tumor from forming or retards, prevents, or alleviates a symptom of the cancer. Assessment of cancer is made using standard clinical protocols. Similarly, increased immunization clinical benefit is determined for example by decreased physician visits, and decreased disease burden in the community.

Methods of Increasing Antibody Secretion

The invention provides a method of increasing or stimulating production and/or secretion of antibodies in a cell. The cell an antibody forming cell such as a B-cell. Alternatively, the cell is a cell that augmenst antibody production by a B cell such as a T-cell (Th and Tc), macrophage, dendritic cell

Antibody secretion by a cell is increased by contacting the cell with the vaccine including an adjuvant polypeptide and an antigen. Antibody secretion by a cell can be increased directly, such as by stimulating B cells, or indirectly, such as by stimulating T cells (e.g., helper T cells), which activated T cells then stimulate B cells. Increased antibody production and/or secretion is measured by methods known to those of ordinary skill in the art, including ELISA, the precipitin reaction, and agglutination reactions.

Methods of Increasing Immune Cell Activation

The invention provides a method of activating or stimulating an immune cell (e.g., a B cell or a T cell). T cell activation is defined by an increase in calcium mediated intracellular cGMP, or an increase in cell surface receptors for IL-2. For example, an increase in T cell activation is characterized by an increase of calcium mediated intracellular cGMP and or IL-2 receptors following contacting the T cell with the vaccine, compared to in the absence of the vaccine. Intracellular cGMP is measured, for example, by a competitive immunoassay or scintillation proximity assay using commercially available test kits. Cell surface IL-2 receptors are measured, for example, by determining binding to an IL-2 receptor antibody such as the PC61 antibody. Immune cell activation can also be determined by measuring B cell proliferative activity, polyclonal immunoglobulin (Ig) production, and antigen-specific antibody formation by methods known in the art.

Pharmeaceutical Compositions

The fusion peptides of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal or patch routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, peptide, or nucleic acid molecule, other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements, which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. a vaccine or fusion protein, in a vector by expression from encoding DNA in a viral vector (see for example, EP-A-415731 and WO 90/07936).

In a specific embodiment of the present invention, nucleic acids include a sequence that encodes a vaccine, or functional derivatives thereof, are administered to modulate immune cell activation by way of gene therapy. In more specific embodiments, a nucleic acid or nucleic acids encoding a vaccine or fusion protein, or functional derivatives thereof, are administered by way of gene therapy. Gene therapy refers to therapy that is performed by the administration of a specific nucleic acid to a subject. In this embodiment of the present invention, the nucleic acid produces its encoded peptide(s), which then serve to exert a therapeutic effect by modulating function of the disease or disorder. Any of the methodologies relating to gene therapy available within the art may be used in the practice of the present invention. See e.g., Goldspiel, et al., 1993. Clin Pharm 12: 488-505.

In a preferred embodiment, the Therapeutic comprises a nucleic acid that is part of an expression vector expressing any one or more of the vaccines, fusion proteins, or fragments, derivatives or analogs thereof, within a suitable host. In a specific embodiment, such a nucleic acid possesses a promoter that is operably-linked to coding region(s) of a fusion protein. The promoter may be inducible or constitutive, and, optionally, tissue-specific. In another specific embodiment, a nucleic acid molecule is used in which coding sequences (and any other desired sequences) are flanked by regions that promote homologous recombination at a desired site within the genome, thus providing for intra-chromosomal expression of nucleic acids. See e.g., Koller and Smithies, 1989. Proc Natl Acad Sci USA 86: 8932-8935.

Delivery of the Therapeutic nucleic acid into a patient may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. In a specific embodiment of the present invention, a nucleic acid is directly administered in vivo, where it is expressed to produce the encoded product. This may be accomplished by any of numerous methods known in the art including, e.g., constructing the nucleic acid as part of an appropriate nucleic acid expression vector and administering the same in a manner such that it becomes intracellular (e.g., by infection using a defective or attenuated retroviral or other viral vector; see U.S. Pat. No. 4,980,286); directly injecting naked DNA; using microparticle bombardment (e.g., a “Gene Gun®; Biolistic, DuPont); coating the nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis (see, e.g., Wu and Wu, 1987. J Biol Chem 262: 4429-4432), which can be used to “target” cell types that specifically express the receptors of interest, etc.

An additional approach to gene therapy in the practice of the present invention involves transferring a gene into cells in in vitro tissue culture by such methods as electroporation, lipofection, calcium phosphate-mediated transfection, viral infection, or the like. Generally, the method of transfer includes the concomitant transfer of a selectable marker to the cells. The cells are then placed under selection pressure (e.g., antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including, e.g. transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. See e.g., Loeffler and Behr, 1993. Meth Enzymol 217: 599-618. The chosen technique should provide for the stable transfer of the nucleic acid to the cell, such that the nucleic acid is expressible by the cell. Preferably, the transferred nucleic acid is heritable and expressible by the cell progeny.

In preferred embodiments of the present invention, the resulting recombinant cells may be delivered to a patient by various methods known within the art including, e.g., injection of epithelial cells (e.g., subcutaneously), application of recombinant skin cells as a skin graft onto the patient, and intravenous injection of recombinant blood cells (e.g., hematopoietic stem or progenitor cells). The total amount of cells that are envisioned for use depend upon the desired effect, patient state, and the like, and may be determined by one skilled within the art.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (International Patent Publication WO 94/08598), neural stem cells (Stemple and Anderson, 1992, Cell 71: 973-985), hematopoietic stem or progenitor cells, e.g., as obtained from bone marrow, umbilical cord blood, peripheral blood, fetal liver, and the like. In a preferred embodiment, the cells utilized for gene therapy are autologous to the patient.

The vaccines of the present invention also include one or more adjuvant compounds. Adjuvant compounds are useful in that they enhance long term release of the vaccine by functioning as a depot. Long term exposure to the vaccine should increase the length of time the immune system is presented with the antigen for processing as well as the duration of the antibody response. The adjuvant compound also interacts with immune cells, e.g., by stimulating or modulating immune cells. Further, the adjuvant compound enhances macrophage phagocytosis after binding the vaccine as a particulate (a carrier/vehicle function).

Adjuvant compounds useful in the present invnetion include Complete Freund's Adjuvant (CFA); Incomplete Freund's Adjuvant (IFA); Montanide ISA (incomplete seppic adjuvant); Ribi Adjuvant System (RAS); TiterMax; Syntex Adjuvant Formulation (SAF); Aluminum Salt Adjuvants; Nitrocellulose-adsorbed antigen; Encapsulated or entrapped antigens; Immune-stimulating complexes (ISCOMs); and Gerbu^{R} adjuvant.

### EXAMPLE 1

### Expression of the Mucin-type (PSGL-1/mIgG_{2B}) and α₁-Acid Glycoprotein (AGP/mIgG_{2B}) Fusion Proteins in the Yeast Pichia Pastoris

The cDNA sequence for a fusion protein comprised of the extracellular part of the mucin-like protein, P-selectin glycoprotein ligand-1, or the whole coding sequence except the translational stop for α₁-acid glycoprotein, and the Fc part of mouse IgG_{2b} will be subcloned into an expression vector for P. pastoris. PSGL-1/mIgG_{2b} carries mainly O-glycans whereas AGP/mIgG_{2b} is exclusively N-glycosylated. The yeast will be transfected and stable transfectants selected using Zeocin as selection drug. Secreted fusion protein will be purified by affinity chromatography and gel filtration, and O- and N-glycans released by β-elimination and PNGase F digestion, respectively. Released saccharides will be characterized by mass spectrometry. The focus of the structural characterization will be on O-glycans, because they have not been characterized in great detail before and our long-term goal is to engineer P. pastoris into synthesizing more human-like O-glycans.

### EXAMPLE 2

### Assess the Ability of Pichia Pastoris-Produced PSGL-1/mIgG_{2B} and AGP/mIgG_{2B} to Bind Mannose Receptors of Macrophages and Dendritic Cells as Well as Mannose Receptors in Serum

Immunoglobulin fusion proteins of PSGL-1 and AGP produced in wild type Pichia will be purified and used in experiments to assess macrophage receptor binding. To this end, isolated macrophages and dendritic cells will be used to assess the ability of mannosylated fusion proteins to promote uptake of fluorescent nano- and microparticles and proteins (i e. green fluorescent protein) after they have been covalently linked to these tracer particles and proteins. Likewise, the effect of mannosylation on the immunogenicity of a model protein will be tested following its conjugation to the mannosylated fusion proteins, uptake by antigen presenting cells (MØ and DCs), and subsequent incubation with purified CD4⁻ and CD8⁺ T lymphocyte populations. Similarly, mannan-binding lectins (MBL) from serum will be tested with regard to their ability to bind the various fusion proteins produced in Pichia. We thereby hope to get some information as to which mannose structures (N- or O-linked) that are important for binding to MBL.

### EXAMPLE 3

### Humanize the Repertoire of O-Glycans Produced by the Yeast Pichia Pastoris

The next step will be to express PSGL-1/mIgG_{2b} with a humanized O-glycan repertoire. To this end, we will co-express one or several UDP-N-acetyl-D-galactosaminide:polypeptide N-acetylgalactosaminyltransferases (ppGalNAc-Ts), which are the enzymes that in a peptide sequence-specific manner adds N-acetylgalactosamine residues to the amino acids serine or threonine in the peptide chain. Initially we will express the native forms of the enzymes. If this results in incorrect ER/Golgi localization, we will express chimeric forms of the enzymes in which the catalytic domain of the ppGalNAc-T has been fused to the transmembrane domain of the yeast-specific mannosyltransferase that links the first mannose residue to the peptide chain. If this does not work, transmembrane signal sequences from other type II proteins in Pichia will be tried. In addition, we most likely need to silence the expression of various mannosyltransferases involved in the biosynthesis of Pichia O-glycans. If a complete silencing through homologous recombination is lethal, we will try to accomplish a partial gene silencing using the siRNA technology. A partial silencing of the endogenous mannosyltransferases may with preserved yeast viability shift the equilibrium enough to favour the transfer of GalNAc residues instead of mannose residues. Further, to obtain a human-like O-glycan repertoire in Pichia it may also be necessary to express the transporter that takes UDP-GalNAc across the Golgi membrane. Mutant yeast colonies carrying human glycosyltransferases will be identified by lectin blots. In brief, replicas of the growing yeast colonies will be made by overlaying them with nitrocellulose membranes in order to capture secreted PSGL-1/mIgG fusion proteins. Following washing, the membranes will be probed with lectins of known carbohydrate specificity. Yeast colonies with the desired glycans on the PSGL-1 Ig fusion will be further expanded, and the O-glycan repertoire carried by the fusion protein will be structurally characterized following its purification. The recombinant protein is purified and structurally characterized as described above. If the initiating glycosylation step is successful, the innermost sugar can be built upon by introducing additional glycosyltransferase genes such that epitopes of therapeutic potential can be made.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A fusion polypeptide comprising a first polypeptide operably linked to a second polypeptide wherein the first polypeptide is mannosylated and the second polypeptide comprises at least a region of an immunoglobulin polypeptide.

2. The fusion polypeptide of claim 1, wherein the first polypeptide is a mucin polypeptide.

3. The fusion polypeptide of claim 2, wherein the mucin is selected from the group consisting of PSGL-1, MUC1, MUC2, MUC3a, MUC3b, MUC4, MUC5a, MUC5b, MUC5c, MUC6, MUC10, MUC11, MUC12, MUC13, MUC15, MUC16, MUC17, CD34, CD43, CD45, CD96, GlyCAM-1, MAdCAM, or a fragment thereof

4. The fusion polypeptide of claim 2, wherein said mucin polypeptide comprises at least a region of a P-selectin glycoprotein ligand-1.

5. The fusion polypeptide of claim 2, wherein said mucin polypeptide includes an extracellular portion of a P-selectin glycoprotein ligand-1.

6. The fusion polypeptide of claim 1, wherein the first polypeptide is an alpha glycoprotein polypeptide.

7. The fusion polypeptide of claim 1, wherein the first polypeptide comprises at least a region of an alpha-1-acid glycoprotein.

8. The fusion polypeptide of claim 1, wherein the second polypeptide comprises a region of a heavy chain immunoglobulin polypeptide.

9. The fusion polypeptide of claim 1, wherein said second polypeptide comprises an Fc region of an immunoglobulin heavy chain.

10. An adjuvant composition comprising the fusion polypeptide of claim 1.

11. The adjuvant composition of claim 10, further comprising a polypeptide carrying Galα1,3Gal epitopes.

12. A method of vaccinating a subject in need thereof comprising administering the subject a composition comprising the adjuvant of claim 10 or 11 and an antigen.

13. A yeast cell genetically engineered to produce the fusion polypeptide of claim 1.

14. The yeast cell of claim 13, wherein said cell is Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei.

15. A genetically engineered lower eukaryotic cell producing human-like glycoproteins characterized as having O-linked glycans.

16. The cell of claim 15, where the cell expresses N-acetylgalactosaminyltransferase(s).

17. A recombinant lower eukaryotic cell producing human-like glycoproteins wherein said cell comprises a nucleic acid molecule encoding N-acetylgalactosaminyltransferase(s).

18. The cell of claim 15 or 17, wherein said cell is Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pyperi, Pichia stiptis, Pichia methanolica, Pichia sp., Saccharomyces cerevisiae, Saccharomyces sp., Hansenulapolymorpha, Kluyveromyces sp., Candida albicans, Aspergillus nidulans, or Trichoderma reesei.

19. The cell of claim 15 or 17, wherein said cell does not express one or more enzymes involved in production of high mannose structures.
